(19) United States
(12) Patent Application Publication
(10) Pub. No.: US 2010/0273208 A1
(43) Pub. Date: Oct. 28, 2010

(54) MICROORGANISM TESTING APPARATUS

(76) Inventors: Kei Takenaka, Kashiwa (JP); Hideki Nakamoto, Tokai (JP); Kazuo Takei, Tokai (JP); Masahiro Kurihara, Yokohama (JP); Yuusuke Watanabe, Hitachi (JP); Hisao Saito, Matsudo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/766,948

(22) Filed: Apr. 26, 2010

(30) Foreign Application Priority Data
Apr. 28, 2009 (JP) 2009-109153

(51) Int. Cl. C12Q 1/04; C12M 1/34

(52) U.S. Cl. 435/34; 435/288.7

(57) ABSTRACT

To eliminate the necessity of a dedicated optical system and the flowing of fluorescent microparticles for aligning excitation light with a flat plate-shaped flow cell which internally includes a flow path, a microorganism testing apparatus includes: a first detector that detects fluorescence emitted from microorganisms flowing through a detection flow path when a microorganism detection unit included in a microorganism testing chip is irradiated with excitation light, and converts the fluorescence to an electrical signal; and a second detector that detects scattered light similarly emitted from the microorganisms flowing through the detection flow path, and converts the scattered light to an electrical signal. The alignment of the detection flow path is performed in the direction of the optical axis of the excitation light by controlling and moving a stage having the microorganism testing chip mounted thereon based on the intensity of fluorescence detected by the first detector.

### BACKGROUND OF THE INVENTION

1. Field of the Invention

The present invention relates to a microorganism testing apparatus.

2. Related Art

Conventionally, there have been known measuring apparatuses that execute various kinds of methods for quick and simple measurement of the number of viable bacteria. In particular, microorganism measuring apparatuses using a fluorescence flow cytometry method have been known as an approach for quick and direct measurement of the number of viable bacteria.

In the fluorescence flow cytometry method, the flow diameter of a fluid specimen containing microorganisms such as bacteria or plankton dyed with fluorochrome is set small, and microorganisms flowing through a flow path are measured one by one. Some of microorganism measuring apparatuses using this method can measure ten thousand or more microorganisms per minute one by one. Further, microorganism measuring apparatuses using this method employ a technique in which a fluid specimen and a sheath liquid are caused to flow in layers to prevent components contained in the specimen from adhering to the wall of a flow path, and in which the flow diameter of the specimen is narrowed down by making use of the pressure difference between the two liquids.

Further, in order to reduce the cost of a microorganism measuring apparatus using this method and eliminate a process for cleaning the microorganism measuring apparatus, a technique has been known in which measurement is performed in a disposable flow path portion prepared as a flow path portion for performing measurement based on the fluorescence flow cytometry method (see Non-Patent Document 1).

In the fluorescence flow cytometry method, it is important to align a flow path for detection with excitation light for the excitation of a specimen. In general, the alignment of the flow path for detection with the excitation light in the fluorescence flow cytometry method is performed by passing fluorescent microparticles serving as marks through the flow path for detection and using as a marker fluorescence emitted by the fluorescent microparticles.

There are examples in which alignment is performed without passing marks through the flow path for detection. For example, Patent Document 1 describes a particle analyzer in which specimen particles flowing through a flow cell made of a rectangular glass tube is irradiated with a laser beam, and in which scattered light and fluorescence emitted by the specimen particles are measured. Specifically, a particle analyzer is described in which a laser beam emitted from a light source for focus detection is projected in the flow cell through an aperture mask having a slit shifted from the center of the optical path to one side, and in which a focusing state is detected by sensing reflected light from front and rear surfaces of the flow cell.

Further, for example, Patent Document 2 describes a flow cytometer using a flat plate-shaped flow cell. The flat plate-shaped flow cell has a structure internally including a rectangular flow path formed by bonding two flat plates to each other, one of which has a grooved structure. In the case of this apparatus, a light beam from an excitation light source is applied to the flat plate-shaped flow cell, and intense scattered light is emitted when the light beam crosses the rectangular flow path in the flat plate-shaped flow cell. Accordingly, in this apparatus, a relationship between the power of scattered light and the position of the flat plate-shaped flow cell or the light beam is predetermined, and alignment is performed by moving the flat plate-shaped flow cell or the light beam.

### Citation List

### Patent Documents

### Patent Document 1: Japanese Patent Application Publication No. 62-91836

### Patent Document 2: Japanese Patent Application Publication No. 2004-69634

### Non-Patent Document

### Non-Patent Document 1: Journal of Biomolecular Techniques, Vol. 14, Issue 2, pp. 119-127

### SUMMARY OF THE INVENTION

A microorganism measuring apparatus using a fluorescence flow cytometry method irradiates a very narrow flow path for detection with excitation light, and detects fluorescence emitted by fine particles to be measured. Accordingly, the flow path needs to be aligned with the focal points of the excitation light and a detector.

However, the ranges of the focal points of the excitation light and the detector and the width of the flow path are on the order of several micrometers to several hundred micrometers. Thus, alignment with an accuracy of several tens of micrometers is necessary for high-accuracy measurement.

In particular, in the case where a disposable flow path portion is used, the alignment of the flow path with the excitation light is needed for each measurement. This leads the inventor to consider that a simple alignment method is necessary.

Further, the method in which fluorescent microparticles are passed through the flow path for detection and in which alignment is performed using fluorescence emitted by fluorescent microparticles as a marker is a general method as a fluorescence flow cytometry method as described previously. However, fluorescent microparticles may adhere to the wall of the detection flow path, and thereby increase background light. In such a case, since weak fluorescence emitted by fine particles to be measured cannot be detected, measurement sensitivity is expected to decrease. Moreover, since fluorescent microparticles for alignment are consumed for each measurement, cost per measurement also increases.

In the case of the method described in Patent Document 1, alignment in the direction of the optical axis of excitation light is performed by sensing reflected light from the front and rear surfaces of the flow cell. This method needs not only an optical system for microorganism detection including a light source, a lens, an optical filter, an optical detector, and the like, but also an optical system for focusing including a light source, a lens, an aperture, an optical array sensor, and the like. This complicates the configuration of an apparatus and also increases fabrication cost.

Moreover, in general, in the case where a disposable flow path is used, a flat plate-shaped flow cell internally including a rectangular flow path formed by bonding two flat plates to each other, one of which has a grooved structure, is often used as the flow path for detection because of the ease of fabrication thereof. For example, in the case of the method described in Patent Document 2, the intensity of scattered light from the flow path rapidly increases when the excitation light crosses the flow path. Accordingly, the alignment of the flow path with the excitation light can be performed in a direction perpendicular to the optical axis of the excitation light. However, when the flow path is moved in a direction parallel to the optical axis, the intensity of the scattered light hardly changes. Thus, in the method described in Patent Document 2, the alignment of the flow path with the excitation light based on a change in the intensity of scattered light is difficult to perform in a direction parallel to the optical axis.

The present invention has been made in view of these circumstances, and an object of the present invention is to provide a microorganism testing apparatus using a fluorescence flow cytometry method. This microorganism testing apparatus is capable of performing the alignment of a flow path for detection (microorganism detection unit) with excitation light in a direction parallel to the optical axis of the excitation light without the installation of an optical system dedicated for alignment and the flowing of fluorescent microparticles.

In order to achieve the above-described object, the present invention provides a microorganism testing apparatus using a fluorescence flow cytometry method. In the microorganism testing apparatus, a stage holding a microorganism testing chip having a microorganism detection unit provided therein is controlled and moved in the direction of the optical axis of excitation light based on the intensity of fluorescence detected from the microorganism detection unit, and the alignment of the microorganism detection unit (flow path for detection) is performed in the direction of the optical axis of the excitation light.

Further, in order to achieve the above-described object, in the present invention, a mark which changes the intensity of fluorescence in accordance with a change in a relative positional relationship with the applied excitation light is provided at a predetermined position (having a fixed positional relationship with the microorganism detection unit (flow path for detection)) in the microorganism testing chip having the microorganism detection unit (flow path for detection) provided therein. In the alignment of the microorganism detection unit (flow path for detection), the stage holding the microorganism testing chip is controlled and moved in the direction of the optical axis of the excitation light based on the intensity of fluorescence detected by applying the excitation light to this mark. Thus, the alignment of the microorganism detection unit (flow path for detection) is performed in the direction of the optical axis of the excitation light.

The intensity of fluorescence detected from the microorganism detection unit (flow cell) depends on the intensity of fluorescence emitted from each point in space and on the light collection efficiency of an optical system for light emitted from each point in space. Accordingly, the higher the intensity of fluorescence emitted from points in space for which light collection efficiency is higher, the higher the intensity of fluorescence detected by a detection device. Further, a member constituting the microorganism detection unit (flow cell) has a higher fluorescence quantum yield than air, and light collection efficiency is higher for light spots closer to the focal point of the optical system. Accordingly, the intensity of fluorescence detected by the detector is maximum when the microorganism detection unit (flow cell) is positioned near the focal point of the excitation light.

For the above-described reasons, the intensity of fluorescence detected from the microorganism detection unit (flow cell) can be related to the position of the microorganism detection unit (flow cell) with respect to the excitation light, and the position of the microorganism detection unit (flow cell) can be adjusted. At this time, fluorescence from the microorganism detection unit (flow cell) can be detected by an optical system for detecting microorganisms. This eliminates the necessity of an optical system dedicated for alignment, and prevents the complication of a device and an increase in device cost. Moreover, since fluorescent microparticles are not used in alignment, it is possible to prevent a decrease in measurement sensitivity due to the adhesion of fluorescent microparticles to the wall of the flow path, and also prevent an increase in measurement cost due to the use of fluorescent microparticles.

These effects are similarly obtained in the case where a mark is provided at a predetermined position in the microorganism testing chip, and where alignment is performed based on the intensity of fluorescence detected by irradiating this mark with the excitation light. In this case, it is necessary to provide the mark of the microorganism testing chip. However, a structure can be employed which increases the intensity of fluorescence without adversely affecting microorganism testing, and a change in fluorescence at the time of alignment can be reliably detected.

### BRIEF DESCRIPTION OF THE DRAWINGS

FIG. 1 is a view schematically showing the configuration of a microorganism testing apparatus according to an embodiment of the present invention.

FIG. 2A is a view showing an example of a cross section of a microorganism testing chip according to the embodiment of the present invention, the cross section including a microorganism detection flow path.

FIG. 2B is an exploded view showing an example of the structure of the microorganism testing chip according to the embodiment of the present invention, the exploded view including the microorganism detection flow path.

FIG. 3 is a view showing an example of a procedure for the alignment of the microorganism testing chip in the microorganism testing apparatus according to the embodiment of the present invention.

FIG. 4 is a schematic diagram for explaining a mechanism for alignment using scattered light according to the embodiment of the present invention.

FIG. 5 is a view for explaining the relationship between the intensity of scattered light and the displacement of the microorganism detection flow path in the embodiment of the present invention.

FIG. 6 is a schematic diagram for explaining a mechanism for alignment using fluorescence according to the embodiment of the present invention.

FIG. 7 is a view for explaining the relationship between the intensity of fluorescence and the displacement of the microorganism detection flow path in the embodiment of the present invention.

FIG. 8 is a view for explaining another example of the structure around the microorganism detection flow path in the microorganism testing chip.

FIG. 9 is a view for explaining an example of an analysis process in the microorganism testing apparatus.

FIG. 10 is a view schematically showing the configuration of the microorganism testing chip according to the embodiment of the present invention.

FIG. 11 is a view showing an example of a schematic configuration of a detection device provided in the microorganism testing apparatus according to the embodiment of the present invention.

### DESCRIPTION OF THE PREFERRED EMBODIMENTS

Hereinafter, an embodiment of the present invention will be described with reference to the drawings. It should be noted that the undermentioned embodiment of the present invention is one example, and other embodiments can be achieved by combination or replacement with publicly- or well-known techniques.

### (A) Example of Overall Configuration of Device

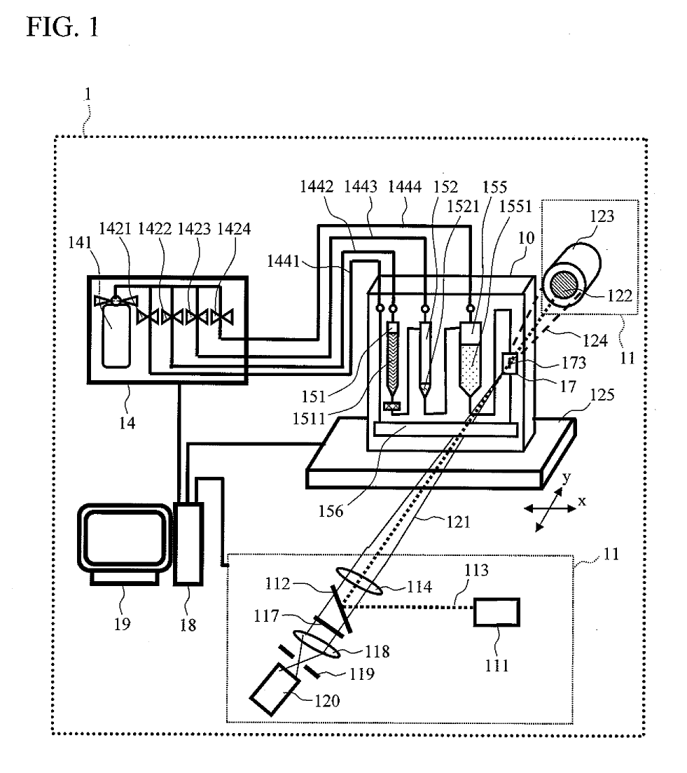

FIG. 1 shows a configuration diagram of a microorganism testing apparatus 1 according to the embodiment of the present invention. The microorganism testing apparatus 1 includes a microorganism testing chip 10, a pressure supply device 14, an X-Y movable stage 125, and a detection device 11. The microorganism testing chip 10 holds a specimen and a reagent therein, and internally includes a mechanism for performing processes necessary for measuring microorganisms. The pressure supply device 14 controls the transportation of a specimen and a reagent in the microorganism testing chip 10 through chip connection pipes 1441-1444 connected to the microorganism testing chip 10 in order to perform processes necessary for measuring microorganisms. The X-Y movable stage 125 holds the microorganism testing chip 10 and adjusts the position of the microorganism testing chip 10. The detection device 11 irradiates microorganisms in the microorganism testing chip 10 with excitation light, and converts scattered light and fluorescence from the microorganisms to an electrical signal. A computer 18 connected to the microorganism testing apparatus 1 outputs control signals to the pressure supply device 14 and the X-Y movable stage 125, and executes the signal processing of an electrical signal inputted from the detection device 11. It should be noted that a measurement result obtained by the processing of the electrical signal is displayed on an output device 19 connected to the computer 18.

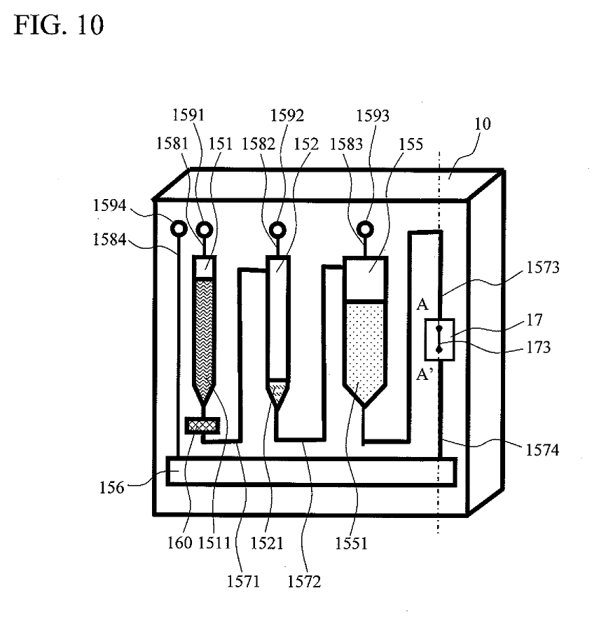

The pressure supply device 14 includes a cylinder 141 with a pressure adjuster. Air, an inert gas, or the like at high pressure is encapsulated in the cylinder 141. The cylinder 141 is connected to ventilation ports 1591 to 1594 (FIG. 10) of the microorganism testing chip 10 by the chip connection pipes 1441 to 1444. The chip connection pipes 1441 to 1444 have valves 1421 to 1424, respectively. By opening or closing the valves 1421 to 1424, containers of the microorganism testing chip 10 are supplied with gas having a predetermined pressure or opened to the atmosphere. This pressure control realizes the transportation of a specimen and a reagent in the microorganism testing chip 10.

The microorganism testing chip 10 includes a specimen container 151, a microorganism dyeing reagent container 152, a diluent container 155, a microorganism detection unit 17, a detection liquid waste container 156, solution flow paths 1571 to 1574, and air flow paths 1581 to 1584. The specimen container 151 holds a specimen 1511. The microorganism dyeing reagent container 152 holds a dyeing reagent (reagent solution) 1521 for dyeing microorganisms in the specimen so that the specimen may be mixed and reacted with the dyeing reagent. The diluent container 155 holds a diluent 1551 so that a liquid mixture of the specimen and the dyeing reagent may be diluted. The microorganism detection unit 17 internally includes a microorganism detection flow path 173 for observing microorganisms with excitation light 113 applied thereto from an excitation light source 111. The detection liquid waste container 156 is used to discard the liquid mixture of the specimen 1511 and the dyeing reagent 1521 which has passed through the microorganism detection flow path 173. The solution flow paths 1571 to 1574 (FIGS. 8 and 10) connect the specimen container 151, the microorganism dyeing reagent container 152, the diluent container 155, and the microorganism detection flow path 173 so that the specimen 1511 and the liquid mixture may flow. The air flow paths 1581 to 1584 (FIG. 10) connect the pressure supply device 14 and the containers, respectively, so that the specimen 1511 and the liquid mixture may flow by the influence of the atmospheric pressure. In this specification, along the flow of the fluid specimen, the specimen container 151 side is defined as an upstream side, and the microorganism detection flow path 173 side is defined as a downstream side.

The detection device 11 includes the excitation light source 111, a scattered light detection unit (corresponding to the “second detector” of the appended claims), and a fluorescence detection unit (corresponding to the “first detector” of the appended claims). Of these, the scattered light detection unit includes a scattered light detector 123 and a shield plate 122. The scattered light detector 123 is intended to detect scattered light 124 from microorganisms passing through the microorganism detection flow path 173. The shield plate 122 is intended to prevent the excitation light 113 from the excitation light source 111 from being incident directly on the scattered light detector 123. On the other hand, the fluorescence detection unit includes an objective lens 114, a dichroic mirror 112, a bandpass filter 117, a condenser lens 118, a pinhole 119, and an optical detector 120. The objective lens 114 focuses fluorescence 121 which has been emitted from microorganisms and has passed through the microorganism detection flow path 173 into a parallel beam. The dichroic mirror 112 reflects the excitation light 113 in the direction of the microorganism detection unit 17 but transmits the fluorescence 121. The bandpass filter 117 transmits the fluorescence 121. The condenser lens 118 focuses the parallel beam. The pinhole 119 is used as a spatial filter for cutting stray light. The optical detector 120 detects light which has passed through the bandpass filter 117. It should be noted that the irradiation unit and the detection unit are disposed such that the focal points thereof coincide with each other, and are configured such that the microorganism detection flow path 173 can be adjusted to the position of the focal points at the time of measurement.

The detection device 11 applies the excitation light 113 outputted from the excitation light source 111 to the microorganism detection flow path 173, and detects the intensity of scattered light produced in the microorganism detection flow path 173 and the intensity of fluorescence produced in the microorganism detection unit 17, thus obtaining as a profile the relationship between each of some possible positions of the X-Y movable stage 125 and the light intensity. The detection device 11 controls and moves the X-Y movable stage 125 based on the obtained profile to adjust the microorganism testing chip 10 (specifically, the microorganism detection flow path 173) to a position suitable for detection. Specifically, the profile obtained by the detection device 11 is stocked in the computer 18, and a control signal is outputted from the computer 18 to the X-Y movable stage 125. Thus, alignment is performed.

### (B) Example of Structure of Microorganism Testing Chip

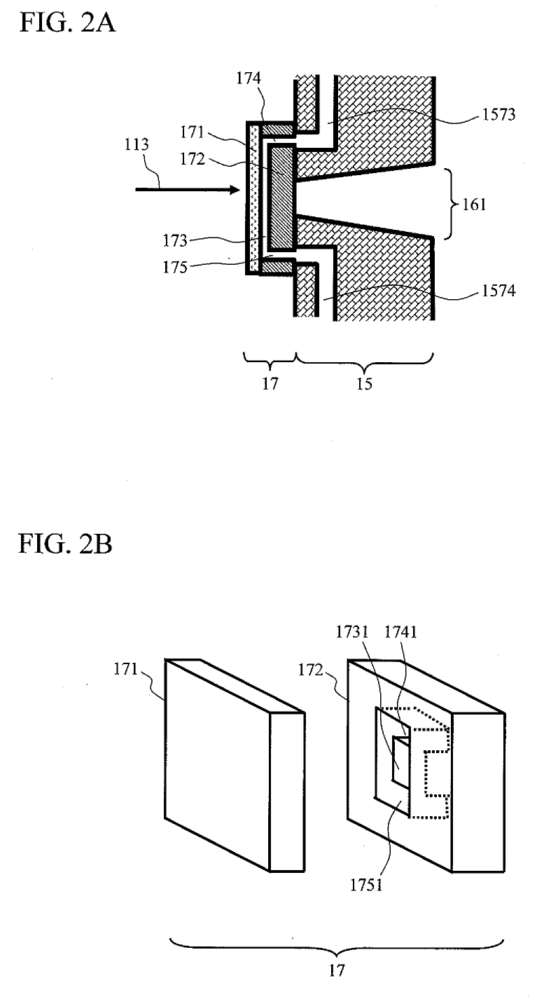

The structure of the microorganism detection unit 17 of the microorganism testing chip 10 will be described with reference to FIGS. 2A and 2B. FIG. 2A shows a cross-sectional view of a joint between a main body 15 of the microorganism testing chip 10 and the microorganism detection unit 17. FIG. 2B shows an exploded perspective view of the microorganism detection unit 17.

It should be noted that the main body 15 and the microorganism detection unit 17 are fabricated in different processes and then joined together. In this embodiment, the microorganism detection unit 17 is configured as a flat plate-shaped flow cell which internally includes flow paths. First, a method of fabricating the microorganism detection unit 17 will be described. The microorganism detection unit 17 includes a cover member 171 and a flow path member 172, both of which are made of thin flat plates. The flow path member 172 has a groove 1731 formed therein, and further has through-holes 1741 and 1751 formed at respective ends of the groove 1731. The cover member 171 and the flow path member 172 are bonded to each other such that the surface of the flow path member 172 which has the groove 1731 formed therein is attached to the cover member 171. By this bonding, the microorganism detection unit 17 is formed. The groove 1731 of the flow path member 172 and the cover member 171 constitute the microorganism detection flow path 173. The through-holes 1741 and 1751 of the flow path member 172 constitute a microorganism detection flow path entrance 174 and a microorganism detection flow path exit 175, respectively.

On the other hand, the flow path between diluent container and microorganism detection flow path 1573 formed in the main body 15 changes the flow path direction thereof at the lower end thereof, and has an opening formed in a surface of the main body 15. Similarly, the flow path between microorganism detection flow path and detection liquid waste container 1574 changes the flow path direction thereof at the upper end thereof, and has an opening formed in the surface of the main body 15. The opening of the flow path between diluent container and microorganism detection flow path 1573 is connected to the microorganism detection flow path entrance 174, and the opening of the flow path between microorganism detection flow path and detection liquid waste container 1574 is connected to the microorganism detection flow path exit 175.

In the main body 15, a detection window frame portion 161 is formed. The detection window frame portion 161 is a through-hole or a pass-through groove. The detection window frame portion 161 is formed between the opening of the flow path between diluent container and microorganism detection flow path 1573 and the opening of the flow path between microorganism detection flow path and detection liquid waste container 1574. The microorganism detection unit 17 fabricated is mounted on the main body 15 as described previously. As shown in FIG. 2A, the microorganism detection unit 17 is mounted such that the microorganism detection flow path 173 is positioned over the detection window frame portion 161 of the main body 15.

In this embodiment, the detection window frame portion 161, which is a through-hole or a pass-through groove, of the main body 15 is provided behind the microorganism detection flow path 173. Accordingly, the excitation light 113 is applied only to the microorganism detection unit 17 and not applied to the main body 15. Thus, reflected light and self-fluorescence, which would cause an increase in background light, are not emitted from the main body 15. To prevent the excitation light 113 which has passed through the microorganism detection flow path 173 from being applied to the main body 15, it is preferable that the area of a cross section of the through-hole constituting the detection window frame portion 161 increase in the direction of emission of the excitation light 113.

The thickness of the cover member 171 is, for example, 0.01 μm to 1 mm. The thickness of the flow path member 172 is, for example, 0.01 μm to 1 mm. The shape of a cross section of the microorganism detection flow path 173 is, for example, square, rectangular, or trapezoidal. The larger the dimensions of the cross section of the microorganism detection flow path 173, the smaller the pressure loss. However, to pass microorganisms through the microorganism detection flow path 173 one by one, it is better that the dimensions of the cross section of the microorganism detection flow path 173 are smaller. Each side of the cross section of the microorganism detection flow path 173 is preferably; for example, 1 μm to 1 mm, and the length of the microorganism detection flow path 173 is preferably, for example, 0.01 mm to 10 mm. The optical axis of the excitation light 113 to be applied to the microorganism detection flow path 173 is perpendicular to the direction vector of the microorganism detection flow path 173.

Materials for the microorganism detection unit 17 will be described. The microorganism testing chip 10 is disposable. In other words, the microorganism detection unit 17 is discarded together with the main body 15 after use. Accordingly, materials for use in the microorganism detection unit 17 should be inexpensive. Materials for use in the microorganism detection unit 17 need to be excellent in optical characteristics so as to be suitable for fluorescence measurement. Specifically, materials are desirable which have low self-fluorescence and which are excellent in optical transparency, profile irregularity, refractive index, and the like. To avoid inhibiting the detection of fluorescence from microorganisms, it is preferable that the intensity of self-fluorescence emitted by the microorganism detection unit 17 itself be sufficiently lower than the intensity of fluorescence from microorganisms.

If the surface of the microorganism detection unit 17 has curved portions, irregularities, or the like, the intensity of the excitation light 113 applied to the microorganism detection flow path 173 fluctuates due to the refraction or diffused reflection of light at the surface. Accordingly, the detected intensity of fluorescence also fluctuates, and measurement accuracy decreases. Thus, the surface of the microorganism detection unit 17 needs to have a desired flatness. The microorganism detection unit 17 preferably has a flatness in which irregularities are 0.1 mm or less.

In consideration of the above-described conditions, possible materials for use in the microorganism detection unit 17 are glass, quartz, polymethacrylic acid methyl ester (PMMA), polydimethylsiloxane (PDMS), cyclo olefin polymer (Cop), polyethylene terephthalate, poly carbonate, and the like. The microorganism detection unit 17 is formed from at least one material selected from these materials.

While the cover member 171 is a simple flat plate, the flow path member 172 is a flat plate having a groove and through-holes formed therein. Accordingly, the flow path member 172 is formed from an easily micromachinable material of low processing cost. Glass and quartz are excellent in optical characteristics but not easy to micromachine. In other words, the micromachining of glass or quartz requires a high processing cost.

Accordingly, in the case of this embodiment, the cover member 171 is formed from glass or quartz, and the flow path member 172 is formed from polymethacrylic acid methyl ester, polydimethylsiloxane, cyclo olefin polymer, polyethylene terephthalate, or poly carbonate. It should be noted that the flow path member 172 is preferably formed from polydimethylsiloxane. In this case, the cover member 171 and the flow path member 172 are joined together by utilizing the self-adhesiveness of polydimethylsiloxane.

The intensity of self-fluorescence of the microorganism detection unit 17 depends not only on materials but also on the thickness dimension of the microorganism detection unit 17. The intensity of self-fluorescence can be reduced by reducing the thickness dimension of the microorganism detection unit 17. The smaller the thickness of the microorganism detection unit 17, the lower the intensity of self-fluorescence emitted from the microorganism detection unit 17. However, if the thickness dimensions of the cover member 171 and the flow path member 172 are reduced, the fabrication thereof becomes difficult, and flatness becomes poor. To maintain a required flatness and reduce the intensity of self-fluorescence to such a level that the detection of fluorescence from microorganisms is not inhibited, the thickness dimensions of these components need to be limited to certain ranges.

The intensities of self-fluorescence of glass, quartz, and polydimethylsiloxane are approximately equivalent. Accordingly, in the case where the cover member 171 is fabricated from glass or quartz, the thickness thereof is preferably, for example, from 0.05 mm to 1 mm, both inclusive. In the case where the flow path member 172 is fabricated from polydimethylsiloxane, the thickness thereof is preferably, for example, from 0.1 mm to 1 mm, both inclusive.

Alternatively, the cover member 171 and the flow path member 172 may be fabricated from cyclo olefin polymer, polymethacrylic acid methyl ester, polyethylene terephthalate, or poly carbonate. In this case, the intensity of self-fluorescence per unit volume is three or more times larger than that in the case where the cover member 171 is fabricated from glass or quartz and where the flow path member 172 is fabricated from polydimethylsiloxane. Accordingly, the thicknesses of the cover member 171 and the flow path member 172 are preferably, for example, from 0.01 mm to 0.3 mm, both inclusive.

### (C) Alignment Operation

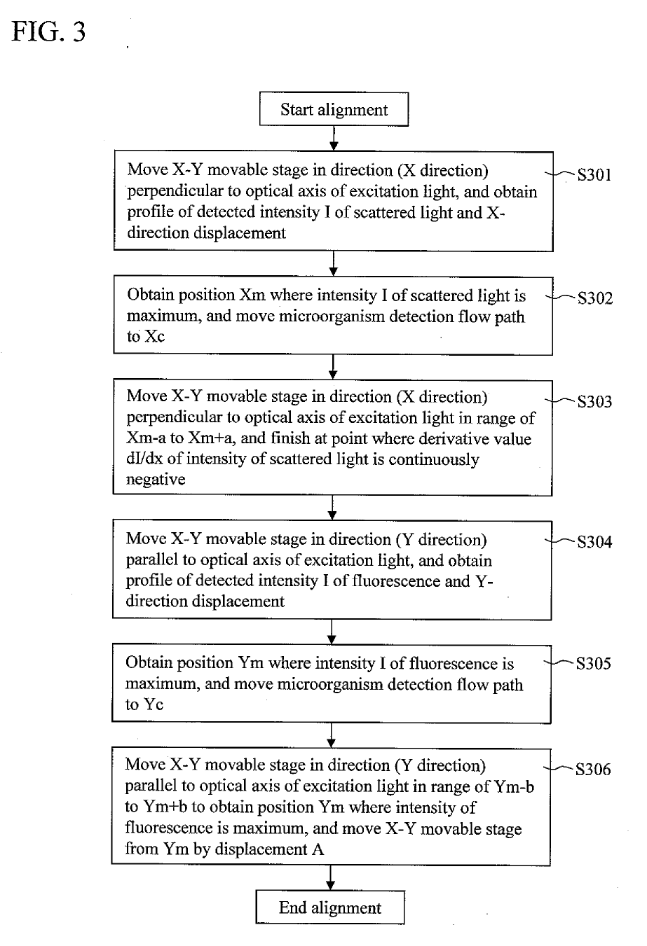

FIG. 3 shows an example of a procedure for aligning the microorganism testing chip 10. The detection device 11 aligns the microorganism detection flow path 173 with the excitation light 113 in accordance with the procedure example shown in FIG. 3.

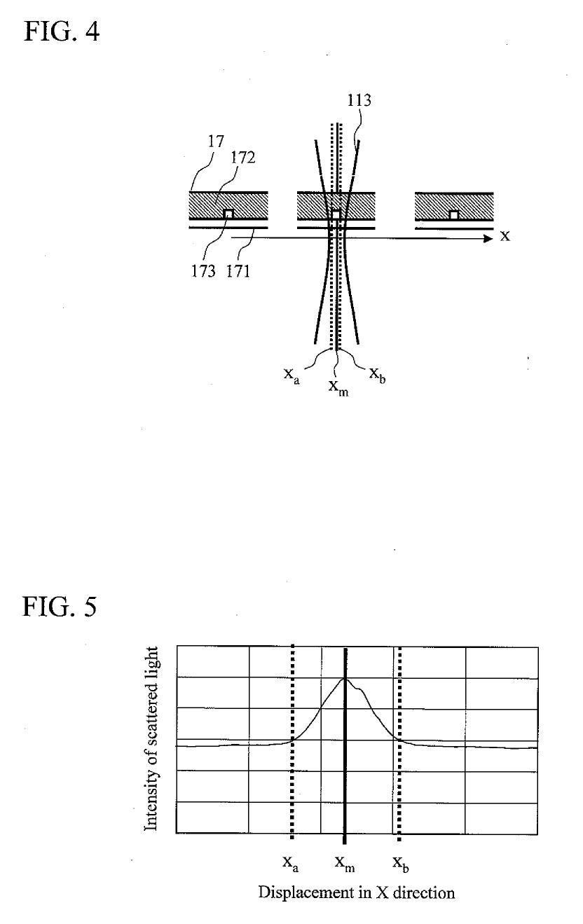

FIGS. 4 to 7 are schematic diagrams for explaining the positioning mechanism of the microorganism detection flow path 173. It should be noted that the cover member 171 is formed from glass, and that the flow path member 172 has a larger thickness than the cover member 171 and is formed from polydimethylsiloxane. Polydimethylsiloxane has a higher self-fluorescence intensity per unit volume than glass.

After the microorganism testing chip 10 is set on the X-Y movable stage 125, the excitation light 113 from the excitation light source 111 is applied to the microorganism detection flow path 173 (S301). Then, using the X-Y movable stage 125, the microorganism testing chip 10 is moved in a direction (X direction) perpendicular to the optical axis of the excitation light 113 (FIG. 4). At this time, the coordinate center of the microorganism detection flow path 173 is denoted by (Xc, Yc).

In this embodiment, alignment in the X direction is performed first, and alignment in the Y direction is then performed. In this embodiment, the alignment in the X direction is performed using a change in the intensity of scattered light from the microorganism detection flow path 173. It should be noted that methods other than this may be used for the alignment in the X direction.

The intensity of the excitation light 113 is highest at the center when the width w (in the case where the excitation light 113 is a laser beam having a Gaussian distribution, the diameter of the range in which the intensity decreases to the value (=center intensity/e²) obtained by dividing the center intensity by e²) of the irradiation range of the excitation light 113 is larger than the width d of the microorganism detection flow path 173. Accordingly, the intensity I of scattered light is highest when the microorganism detection flow path 173 overlap the center of the excitation light 113 (i.e., Xc=Xm). At this time, the relationship between the X-direction displacement of the microorganism detection flow path 173 and the intensity I of scattered light detected by the detection device 11 is such as shown in FIG. 5.

The detection device 11 stocks a profile of the detected intensity I of scattered light and the X-direction displacement in the computer 18. Then, the detection device 11 sets as Xm the position of the center of the microorganism detection flow path 173 where the intensity I of scattered light is maximum, and moves the center of the microorganism detection flow path 173 to Xm (=Xc) (S302). It should be noted that in the alignment, though a shift to the position of Xm (=Xc) is desirable, certain errors are tolerated. Specifically, since there is no problem in microorganism testing as long as the detection flow path is placed within such a range that the intensity of the laser is 80% or more of the maximum in the distribution of intensity of the laser in the X direction, errors in such a range are tolerated. For example, in the case where the range of 80% or more of the maximum in the distribution of intensity of the laser is ±30 μm from the center of the laser beam, when the flow path width is 40 μm, the tolerance is ±10 μm. Alignment is performed so that the center of the microorganism detection flow path 173 may be at least in this range.

To further improve accuracy, the computer 18 performs control based on the detection device 11 so that the X-Y movable stage 125 may be moved in the range of Xm−a to Xm+a in a direction (X direction) perpendicular to the optical axis of the excitation light. This movement of the X-Y movable stage 125 by the detection device 11 is finished at a point where the derivative value dI/dx of the intensity of scattered light is continuously negative (S303).

In the case where the width w of the irradiation range of the excitation light 113 is smaller than the width d of the microorganism detection flow path 173, the intensity of scattered light is maximum when a side surface of the microorganism detection flow path 173 overlaps the center of the excitation light 113. Accordingly, the intensity I of fluorescence detected by the detection device 11 has two peaks with respect to the X-direction displacement. In this case, the detection device 11 sets the midpoint between the two peaks as Xm, and controls and moves the X-Y movable stage 125 so that the microorganism detection flow path 173 may overlap the center of the excitation light 113.

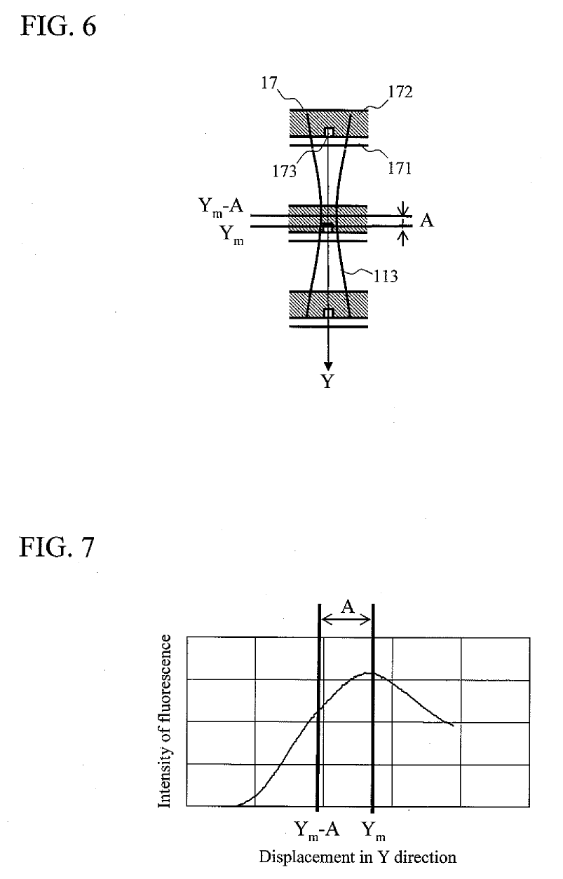

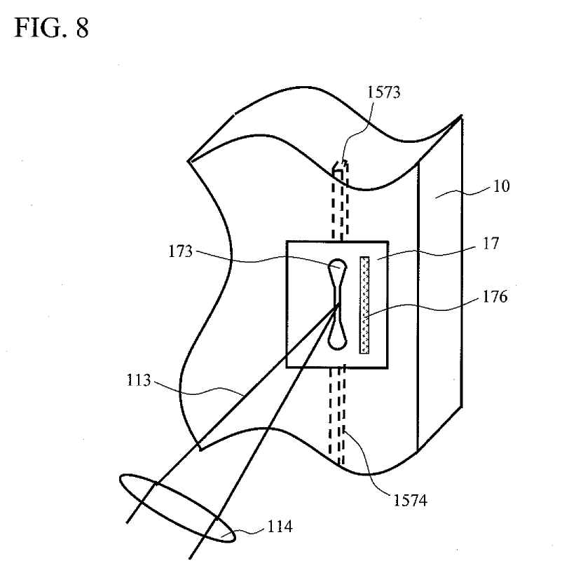

Then, the alignment in the Y direction is performed. In this embodiment, alignment is performed using a change in the intensity of fluorescence from the components of the flow path. The detection device 11 moves the microorganism testing chip 10 in a direction (Y direction) parallel to the optical axis of the excitation light 113 through the movable control of the X-Y movable stage 125 (FIG. 6, S304). The microorganism detection unit 17 emits fluorescence from a portion irradiated with the excitation light 113. At this time, the relationship between the Y-direction displacement and the intensity I of fluorescence detected by the detection device 11 is such as shown in FIG. 7. The intensity of fluorescence emitted from a unit volume is proportional to the fluorescence quantum yields of substances present per unit volume and the intensity of the excitation light 113 applied to a unit volume. Further, the intensity of the excitation light 113 applied to a unit volume is highest near the focal point of the optical system. Moreover, light collection efficiency is higher for light spots closer to the focal point of the optical system of the detection device 11. Accordingly, the intensity I of fluorescence detected by the detection device 11 is highest when a substance having a high fluorescence quantum yield is positioned near the focal point. Polydimethylsiloxane, which is a material for the flow path member 172, has a higher fluorescence quantum yield than glass, which is a material for the cover member 171. Thus, the intensity I of detected fluorescence is highest when the flow path member 172 overlaps the focal point of the excitation light 113.

As in the case of the X direction, the detection device 11 stocks a profile of the intensity I of detected fluorescence and the Y-direction displacement in the computer 18, and sets as (Ym (=Yc)) the position of the center of the microorganism detection flow path 173 where the intensity I of fluorescence is maximum. The distance at this time between the center of the microorganism detection flow path 173 and the focal point of the excitation light 113 is denoted by A (S305). To align the center of the microorganism detection flow path 173 with the focal point of the excitation light 113 more accurately, the detection device 11 moves the microorganism detection flow path 173 to a position obtained by correction using the distance A and represented by Ym−A. Here, the positional relationship between the microorganism detection unit 17 and the focal point of the excitation light 113 when the intensity I of fluorescence is maximum is determined by the distribution of intensity of the excitation light 113, the light collection efficiency of the optical system, the structure of the microorganism detection unit 17, and the fluorescence quantum yields of the components. However, the displacement A may be determined by obtaining the positional relationship between the microorganism detection unit 17 and the focal point of the excitation light 113 when the intensity I of detected fluorescence is maximum by ray tracing using a calculating formula based on the law of refraction. In the case where the flow path member 172 is thin and the displacement A is very small, there is no significant problem in measurement even if correction is not performed.

It should be noted that in the alignment in the Y direction, certain errors are tolerated as in the X direction. Specifically, since there is no problem in microorganism testing as long as Ym is a position where the obtained intensity of fluorescence is 95% or more of the maximum, errors in such a range are tolerated. For example, in the case where the tolerance of Ym in the optical system of this embodiment is ±20 μm and where the flow path depth is 20 μm, alignment tolerance is ±10 μm.

To further improve accuracy, the detection device 11 performs control so that the X-Y movable stage 125 may be moved in the range of Ym−b to Ym+b in a direction (Y direction) parallel to the optical axis of the excitation light. This movement of the X-Y movable stage 125 by the detection device 11 is finished at a position Ym where the intensity I of fluorescence is maximum (S306).

It should be noted that alignment can be performed with higher accuracy by employing an approach in which processing operations for obtaining Xm and Ym are repeated in the X and Y directions, respectively.

### (D) Other Example of Structure of Microorganism Testing Chip

FIG. 8 shows another embodiment of the microorganism detection unit 17 of the microorganism testing chip 10. In the case of FIG. 8, a mark 176 is provided in part of the microorganism detection unit 17. The mark 176 is a structure intended to increase the intensity of scattered light, reflected light, or fluorescence, and is disposed in the vicinity of the microorganism detection flow path 173 to be parallel to the flow path direction. In the case where the microorganism testing chip 10 is moved in a direction (X direction) perpendicular to the optical axis of the excitation light 113 using the X-Y movable stage 125 (FIG. 1), when the mark 176 overlaps the center of the excitation light 113 (Xm), the intensity of scattered light, reflected light, or fluorescence is maximum. The positional relationship between the mark 176 and the microorganism detection flow path 173 with respect to the X direction is known at the design phase. Accordingly, the alignment of the microorganism detection unit in the X direction is performed by moving the microorganism testing chip 10 by a predetermined amount from a position where the intensity of scattered light, reflected light, or fluorescence is maximum. Thus, the microorganism detection flow path 173 can be aligned with the excitation light 113 in a direction perpendicular to the excitation light 113.

Possible structures of the mark 176 which are capable of increasing the intensity of scattered light include, for example, a structure in which an enormous number of fine protrusions or irregularities are formed and a structure in which an enormous number of bubbles or fine particles are internally formed. Similarly, possible structures of the mark 176 which are capable of increasing the intensity of reflected light include, for example, a structure in which a substance having a high reflectance for the wavelength of the excitation light 113 is provided internally or on the surface thereof. Further, possible structures of the mark 176 which are capable of increasing the intensity of fluorescence include a structure in which a substance that is caused to emit fluorescence by the excitation light 113 is provided internally or on the surface thereof.

In the case where the respective positional relationships between the mark 176 and the microorganism detection unit (flow path for detection) with respect to the X and Y directions are accurately determined, alignment can also be performed in the Y direction as in the X direction. Specifically, as to the Y direction, by giving the mark 176 a structure for increasing the intensity of fluorescence (mark that changes the intensity of fluorescence in accordance with a change in a relative positional relationship with applied excitation light), the principle is established that alignment is performed by applying excitation light to the aforementioned detection flow path and detecting fluorescence. The alignment of the microorganism detection unit in the Y direction can be performed by moving the microorganism detection unit by a predetermined amount from a position where the intensity of fluorescence is maximum at the time of the alignment.

### (E) Microorganism Measurement

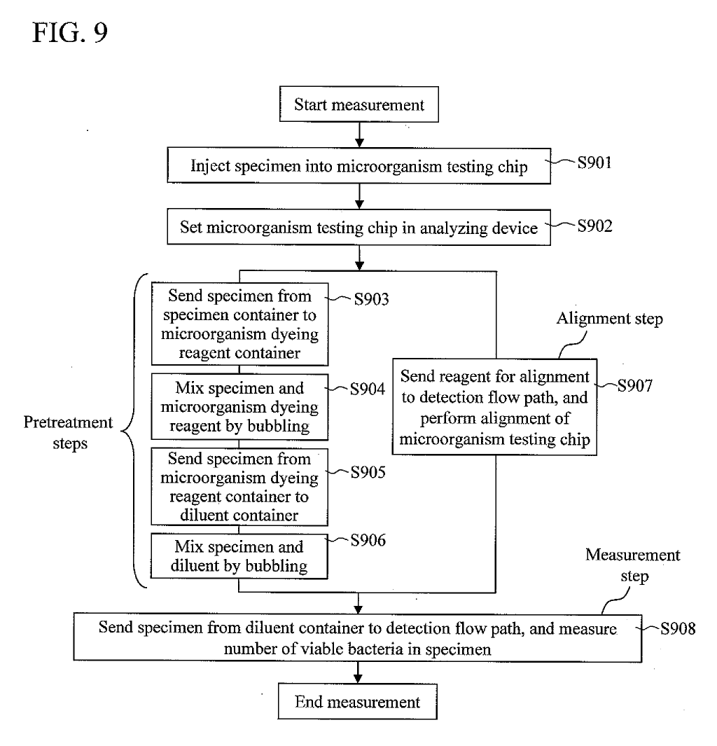

Hereinafter, a description will be made of an example for the case where the number of viable bacteria in a specimen taken from a food is measured using the microorganism testing apparatus 1 according to the embodiment of the present invention. FIG. 9 shows a flowchart of steps for measuring the number of viable bacteria using the microorganism testing chip 10. FIG. 10 shows a plan view of the microorganism testing chip 10.

First, the configuration of the microorganism testing chip 10 will be described. The microorganism testing chip 10 includes the specimen container 151, the microorganism dyeing reagent container 152, the diluent container 155, a food residue removing portion 160, the microorganism detection flow path 173, the detection liquid waste container 156, the solution flow paths 1571 to 1574, the ventilation ports 1591 to 1594, and the air flow paths 1581 to 1584. The specimen container 151 holds the specimen 1511. The microorganism dyeing reagent container 152 holds the dyeing reagent (reagent solution) 1521 for dyeing microorganisms in the specimen. The diluent container 155 holds the diluent 1551. The food residue removing portion 160 is a filter for removing food residues contained in the specimen. The microorganism detection flow path 173 is used to observe fluorescence from microorganisms with excitation light applied thereto from an external light source. The detection liquid waste container 156 is used to discard a liquid mixture of the specimen 1511, the microorganism dyeing reagent 1521, and the diluent 1551 having passed through the microorganism detection flow path 173. The solution flow paths 1571 to 1574 connect the specimen container 151, the food residue removing portion 160, the microorganism dyeing reagent container 152, the diluent container 155, and the microorganism detection flow path 173 so that the specimen 1511 and the liquid mixture may flow. The ventilation ports 1591 to 1594 allow the specimen 1511 and the liquid mixture in the respective containers to flow by the influence of the atmospheric pressure. The air flow paths 1581 to 1584 connect the ventilation ports 1591 to 1594 to the respective containers.

From the names of the containers to be connected, the solution flow paths 1571 to 1574, the ventilation ports 1591 to 1594, and the air flow paths 1581 to 1584 are respectively referred to as the flow path between specimen container and microorganism dyeing reagent container 1571, the flow path between microorganism dyeing reagent container and diluent container 1572, the flow path between diluent container and microorganism detection flow path 1573, the flow path between microorganism detection flow path and detection liquid waste container 1574, the specimen container ventilation port 1591, the microorganism dyeing reagent container ventilation port 1592, the diluent container ventilation port 1593, the detection liquid waste container ventilation port 1594, the specimen container air flow path 1581, the microorganism dyeing reagent container air flow path 1582, the diluent container air flow path 1583, and the detection liquid waste container air flow path 1584.

The specimen container 1511, the food residue removing portion 160, the microorganism dyeing reagent container 152, the diluent container 155, the microorganism detection flow path 173, and the detection liquid waste container 156 are connected in series by the solution flow paths 1571 to 1574.

In the case of FIG. 10, the solution flow paths 1571 to 1574 and the air flow paths 1581 to 1584 are formed to satisfy the following: the depths and widths of the solution flow paths 1571 to 1574 are, for example, in the range of 10 μm to 1 mm; the depths and widths of the air flow paths 1581 to 1584 are, for example, in the range of 10 μm to 1 mm; and the areas of cross sections of the solution flow paths 1571 to 1574 are larger than the areas of cross sections of the air flow paths 1581 to 1584.

The microorganism dyeing reagent 1521 is encapsulated in the microorganism testing chip 10 in advance. The specimen 1511 is injected into the specimen container 151 through the ventilation port 1591 before testing, and the diluent 1551 is injected into the diluent container 155 through the ventilation port 1593 before testing (S901).

The volume of the specimen container 151 is larger than that of the specimen 1511. The volume of the microorganism dyeing reagent container 152 is larger than the total volume of the specimen 1511 and the microorganism dyeing reagent 1521. The volume of the diluent container 155 is larger than the total volume of the specimen 1511, the microorganism dyeing reagent 1521, and the diluent 1551. Further, the flow path between specimen container and microorganism dyeing reagent container 1571 is formed such that the highest point thereof is higher than the water level of the specimen 1511 in the specimen container 151. Similarly, the flow path between microorganism dyeing reagent container and diluent container 1572 is formed such that the highest point thereof is higher than the water level of a liquid mixture of the specimen 1511 and the microorganism dyeing reagent 1521. Moreover, the flow path between diluent container and microorganism detection flow path 1573 is formed such that the highest point thereof is higher than the water level of the liquid mixture of the specimen 1511, the microorganism dyeing reagent 1521, and the diluent 1551.

The specimen 1511 used here is obtained by stomaching a food to be inspected after adding a physiological salt solution which is ten times the food to be inspected by mass. The diluent 1551 is principally a physiological salt solution or pure water.

The microorganism dyeing reagent 1521 is a liquid mixture of a killed bacteria dyeing reagent for dyeing killed bacteria and an all bacteria dyeing reagent for dyeing all bacteria. As the killed bacteria dyeing reagent, for example, PI (propidium iodide) (0.1 μg/ml to 1 mg/ml) is used. As the all bacteria dyeing reagent, for example, DAPI (4′,6-diamidine 2′-phenylindole) (1 μg/ml to 1 mg/ml), AO (acridine orange) (1 μg/ml to 1 mg/ml), EB (ethidium bromide) (1 μg/ml to 1 mg/ml), LDS751 (0.1 μg/ml to 1 mg/ml), or the like is used.

The measurement of the number of viable bacteria using the microorganism testing chip 10 is started with the microorganism testing chip 10 set in the microorganism testing apparatus (analyzing apparatus) 1 as shown in FIG. 9 (S902). This measurement process includes an alignment step (S907) for performing the alignment of the microorganism testing chip 10, pretreatment steps (S903 to 5906) for removing food residues from the specimen and dyeing microorganisms in the specimen, and a measurement step (S908) for actually measuring the number of viable bacteria.

The alignment step (S907) and the pretreatment steps (S903 to 5906) are independent of each other and therefore performed in parallel. After both the alignment step and the pretreatment steps are finished, the measurement step (S908) is performed. Next, the movement of each liquid in each step will be described.

In the pretreatment steps, first, the specimen 1511 is moved to the microorganism dyeing reagent container 152 (S903). Then, pressure from the pressure supply device 14 is applied to the specimen container 151 through the ventilation port 1591. This increases the air pressure in the specimen container 151. At the same time, the microorganism dyeing reagent container 152 is opened to the atmosphere through the microorganism dyeing reagent container ventilation port 1592 so that the internal pressure thereof may be the atmospheric pressure. The difference in air pressure causes the specimen 1511 to enter the microorganism dyeing reagent container 152 and be mixed with the microorganism dyeing reagent 1521. The mixing is performed by bubbling (S904). While killed bacteria in the specimen 1511 are dyed with the killed bacteria dyeing reagent (PI (peak wavelength: 532 nm) is used here) and the all bacteria dyeing reagent (LDS751 (peak wavelength: 710 nm) is used here), viable bacteria in the specimen 1511 are dyed only with the all bacteria dyeing reagent.

The water level of the liquid mixture of the two liquids does not exceed the highest point of the flow path between microorganism dyeing reagent container and diluent container 1572 connecting the microorganism dyeing reagent container 152 and the diluent container 155. Further, air contained in the microorganism dyeing reagent container 152 is discharged to the outside through the microorganism dyeing reagent container ventilation port 1592. The air pressure in the microorganism dyeing reagent container 152 equals to the atmospheric pressure. Accordingly, the liquid mixture of the two liquids is not pushed out to the diluent container 155. Thus, the liquid mixture can be held in the microorganism dyeing reagent container 152 during the time required for the reaction.

At this time, to prevent the liquid mixture from flowing into the diluent container 155, the air pressures in the diluent container 155 and the detection liquid waste container 156 may be increased to pressures lower than the air pressure in the specimen container 151 by applying pressure from the pressure supply device 14 to the ventilation ports 1593 to 1594.

It should be noted that the temperature of the microorganism testing chip 10 is preferably kept constant during dyeing to reduce the influence of temperature change on dyeing.

When the specimen 1511 flows through the food residue removing portion 160 into the microorganism dyeing reagent container 152, food residues in the specimen 1511 are removed from the specimen 1511 by the food residue removing portion 160.

Then, the liquid mixture of the specimen 1511 and the microorganism dyeing reagent 1521 is moved to the diluent container 155 (S905). The diluent 1551 is added to the liquid mixture to reduce the concentration of uncombined dye (microorganism dyeing reagent 1521 which has not dyed microorganisms) contained in the liquid mixture. It should be noted that the mixing of the liquid mixture and the diluent 1551 is performed by bubbling (S906). The reduction of the concentration of uncombined dye reduces the intensity of fluorescence emitted by uncombined dye which causes noise at the time of detection.

This ends the pretreatment steps. In parallel with the pretreatment steps, the alignment of the microorganism testing chip 10 is executed which has been described in the aforementioned embodiment of the present invention.

After the above-described operation is finished, the liquid mixture of the specimen 1511, the microorganism dyeing reagent 1521, and the diluent 1551 is moved to the microorganism detection flow path 173 (S908). In the case of FIG. 10, the excitation light 113 is applied in a direction perpendicular to the drawing plane. Accordingly, there are produced fluorescence from dye which has dyed microorganisms and scattered light from microorganisms. The detection device 11 detects only fluorescence emitted by the all bacteria dyeing reagent for viable bacteria, and detects fluorescence emitted by the all bacteria dyeing reagent and the killed bacteria dyeing reagent for killed bacteria. This enables discrimination between viable bacteria and killed bacteria. Further, since the intensity of scattered light depends on the sizes of fungus bodies, the sizes of fungus bodies can be discriminated.

### (F) Configuration Example of Detection Device

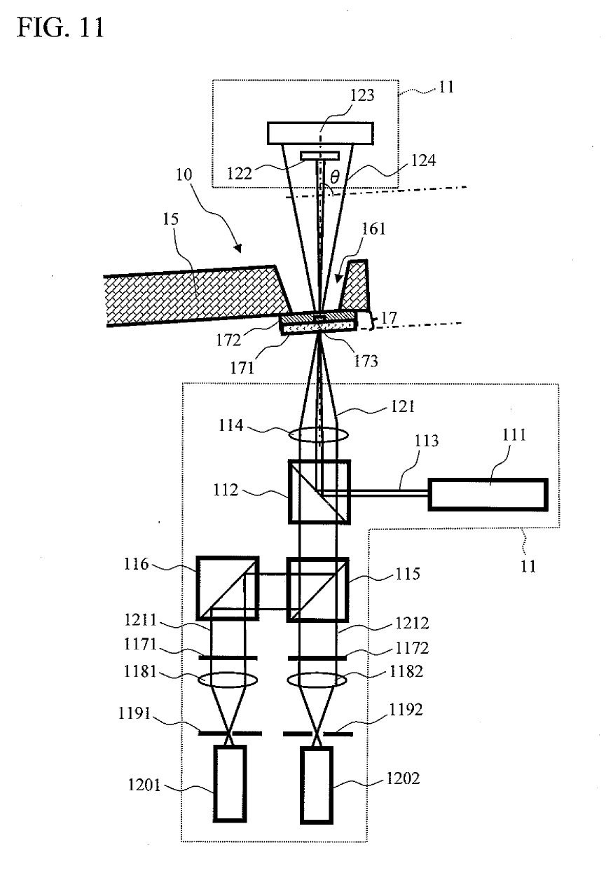

Hereinafter, a description will be made of a configuration example of the detection device 11 included in the microorganism testing apparatus 1 with reference to FIG. 11. As described previously, the detection device 11 is suitable for the measurement of the number of viable bacteria in a specimen taken from a food. In other words, the use of the detection device 11 enables discrimination between the number of viable bacteria and the number of killed bacteria. The optical system of the detection device may differ between the excitation and fluorescence spectra of fluorochrome. Here, a description will be made of the case where PI (excitation wavelength peak: 532 nm, fluorescence wavelength peak: 615 nm) is used as the killed bacteria dyeing reagent and where LDS751 (excitation wavelength peak: 541 nm, fluorescence wavelength peak: 710 nm) is used as the all bacteria dyeing reagent.

The optical system of the detection device 11 in FIG. 11 is configured to be suitable for the case where two types of fluorochromes are used. It is a matter of course that in the case where three or more types of fluorochromes are used, an optical system is prepared for each fluorochrome.

The detection device 11 includes the excitation light source 111 (wavelength: 532 nm), the scattered light detector 123, the shield plate 122, the excitation light-fluorescence separation dichroic mirror 112, the objective lens 114, a fluorescence separation dichroic mirror 115, a mirror 116, short and long wavelength bandpass filters 1171 and 1172, short and long wavelength condenser lenses 1181 and 1182, short and long wavelength pinholes 1191 and 1192, and short and long wavelength optical detectors 1201 and 1202. The scattered light detector 123 detects the scattered light 124 from microorganisms passing through the microorganism detection flow path 173. The shield plate 122 prevents the excitation light 113 from the excitation light source 111 from being incident directly on the scattered light detector 123. The excitation light-fluorescence separation dichroic mirror 112 reflects the excitation light 113 and transmits fluorescence from microorganisms. The objective lens 114 focuses fluorescence which has been emitted from microorganisms and which has passed through the microorganism detection flow path 173 into a parallel beam. The fluorescence separation dichroic mirror 115 reflects light with wavelengths of 610 nm or less and transmits light with wavelengths of 610 nm or more. The short wavelength bandpass filter 1171 transmits only light with wavelengths near 610 nm. The long wavelength bandpass filter 1172 transmits light with wavelengths near 710 nm. The short and long wavelength condenser lenses 1181 and 1182 focus the parallel beam. The short and long wavelength pinholes 1191 and 1192 are used as spatial filters for cutting stray light. The short wavelength optical detector 1201 detects fluorescence 1211 which has passed through the short wavelength bandpass filter 1171. The long wavelength optical detector 1202 detects fluorescence 1212 which has passed through the long wavelength bandpass filter 1172.

As the excitation light source 111, a laser beam source is used. As the scattered light detector, a photodiode is used. As the short and long wavelength optical detectors 1201 and 1202, photomultiplier tubes (PMTs, Photomultipliers) are used. As described previously, the alignment of the microorganism detection flow path 173 of the microorganism testing chip 10 is assumed to be finished. In other words, the microorganism detection flow path 173 of the microorganism testing chip 10 is assumed to be disposed at the position of the focal point of the objective lens 114.

The excitation light 113 (wavelength: 532 nm) outputted from the excitation light source 111 (wavelength: 532 nm) is reflected by the excitation light-fluorescence separation dichroic mirror 112 to change the direction of travel thereof. Thus, the microorganism detection flow path 173 is irradiated with the excitation light 113. This irradiation excites PI and LDS751 which have dyed microorganisms flowing through the microorganism detection flow path 173. Both the fluorescence 1211 (center wavelength of PI: 610 nm) from the killed bacteria dyeing reagent PI and the fluorescence 1212 (center wavelength: 710 nm) from the all bacteria dyeing reagent LDS751 enter the objective lens 114.

While the fluorescence 1211 from the killed bacteria dyeing reagent PI is reflected by the fluorescence separation dichroic mirror 115, the fluorescence 1212 from the all bacteria dyeing reagent LDS751 passes through the fluorescence separation dichroic mirror 115. Thus, the fluorescences originated from two dyes are separated by the difference in wavelength. The fluorescence 1211 from the killed bacteria dyeing reagent PI passes through the short wavelength bandpass filter 1171 to be condensed by the short wavelength condenser lens 1181, and passes through the short wavelength pinhole 1191 to enter the short wavelength optical detector 1201. The fluorescence 1212 from the all bacteria dyeing reagent LDS751 passes through the long wavelength bandpass filter 1172 to be condensed by the long wavelength condenser lens 1182, and passes through the long wavelength pinhole 1192 to enter the long wavelength optical detector 1202.

Moreover, the excitation light 113 outputted from the excitation light source 111 hits microorganisms flowing through the microorganism detection flow path 173 to produce scattered light 124. Since the intensity of the scattered light depends on the sizes of fine particles, the sizes of the fine particles can be measured. If measurement information on the sizes of the fine particles is obtained, microorganisms can be discriminated from undesired substances such as food residues.

Each of fluorescence detected by the short and long wavelength optical detector 1201 and 1202, and scattered light detected by the scattered light detector 123 is converted to an electrical signal and then sent to the computer 18 (FIG. 1). The computer 18 processes the electrical signals sent from the short wavelength optical detector 1201, the long wavelength optical detector 1202, and the scattered light detector 123, and outputs information on the number of microorganisms as a test result to the output device 19 (FIG. 1).

The number of killed bacteria is obtained from the output of the short wavelength optical detector 1201, and the number of all bacteria is obtained from the output of the long wavelength optical detector 1202. Further, the number of viable bacteria is obtained from the difference between the two numbers of bacteria.

Part of the excitation light 113 from the excitation light source 111 may be reflected by the surface of the microorganism detection unit 17 to return to the detection device 11. To prevent this reflection, it is preferable that the normal vector of the microorganism detection unit 17 be not parallel to the optical axis of the excitation light 113. Specifically, the angle α between the normal vector of the microorganism detection unit 17 and the optical axis of the excitation light 113 is preferably α=10 to 20°. FIG. 11 shows an example of such an installation.

In FIG. 11, the angle between the surface of the microorganism detection unit 17 and the optical axis of the excitation light 113 is denoted by 9. The sum θ+α is 90°. The angle θ is less than 90 degrees, and is set such that the total reflection of the excitation light 113 does not occur at the surface of the microorganism detection unit 17. The angle θ may be in the range of 80 to 70°. It should be noted that though the microorganism detection unit 17 is inclined so that the normal vector of the microorganism detection unit 17 may not be parallel to the optical axis of the excitation light 113, the excitation light is applied to the microorganism detection flow path 173 in a direction perpendicular thereto.

### EXPLANATION OF REFERENCE NUMERALS

1 . . . microorganism testing apparatus, 10 . . . microorganism testing chip, 11 . . . detection device, 14 . . . pressure supply device, 17 . . . microorganism detection unit, 18 . . . computer, 19 . . . output device, 111 excitation light source, 113 . . . excitation light, 114 . . . objective lens, 115 . . . fluorescence separation dichroic mirror, 116 mirror, 119 . . . pinhole, 121 . . . fluorescence, 124 . . . scattered light, 125 . . . X-Y movable stage, 151 specimen container, 152 . . . microorganism dyeing reagent container, 155 . . . diluent container, 156 . . . detection liquid waste container, 161 . . . detection window frame portion, 173 . . . microorganism detection flow path, 1171 . . . short wavelength bandpass filter, 1172 . . . long wavelength bandpass filter, 1181 and 1182 . . . condenser lenses, 1191 and 1192 . . . pinholes, 1201 . . . short wavelength optical detector, 1202 . . . long wavelength optical detector, 1211 . . . fluorescence from PI, 1212 . . . fluorescence from LDS751, 1571 to 1574 solution flow paths, 1581 to 1584 . . . air flow paths, 1591 to 1594 . . . ventilation ports

What is claimed is:

1. A microorganism testing apparatus comprising:
a microorganism testing chip including
a specimen container for holding a fluid specimen containing microorganisms,
a reaction container for holding a reagent solution capable of reacting with the fluid specimen and for causing the fluid specimen to react with the reagent solution, and
a microorganism detection unit for detecting the microorganisms;
a pressure supply device for transporting the fluid specimen and the reagent solution within the microorganism testing chip, the pressure supply device being connected to the microorganism testing chip;
a stage for holding the microorganism testing chip and for moving the microorganism testing chip; and
a detection device including
a light source for irradiating the microorganism detection unit with excitation light,
a first detector for detecting fluorescence emitted from microorganisms flowing through a detection flow path of the microorganism detection unit and for converting the fluorescence to an electrical signal, and
a second detector for detecting scattered light emitted from the microorganisms flowing through the microorganism detection unit and for converting the scattered light to an electrical signal,
the detection device controlling a position of the detection flow path in a direction of an optical axis of the excitation light by controlling and moving the stage based on a detected intensity of the fluorescence emitted from the microorganism detection unit, the detected intensity changing depending on a position of the microorganism testing chip.

2. The microorganism testing apparatus according to claim 1, wherein
the microorganism detection unit includes a cover member and a flow path member, and
a groove included in the flow path member forms the detection flow path when the cover member and the flow path member are bonded to each other.

3. The microorganism testing apparatus according to claim 2, wherein
the detection device executes processing for alignment of the detection flow path in a direction perpendicular to the optical axis of the excitation light and processing for alignment of the detection flow path in a direction parallel to the optical axis of the excitation light by using the stage.

4. The microorganism testing apparatus according to claim 3, wherein
the detection device locates the detection flow path at a focal point of the excitation light by obtaining a position of the detection flow path where the intensity of the detected fluorescence is maximum.

5. The microorganism testing apparatus according to claim 3, wherein
the detection device locates the detection flow path on the optical axis of the excitation light by obtaining a position of the detection flow path where the intensity of the detected scattered light is maximum.

6. The microorganism testing apparatus according to claim 3, wherein
the microorganism testing chip includes a mark disposed near the detection flow path, the mark increasing the intensity of scattered light, reflected light, or fluorescence by being irradiated with the excitation light, and
the processing for the alignment of the detection flow path in the direction perpendicular to the optical axis of the excitation light is performed based on scattered light, reflected light, or fluorescence from the mark.

7. A microorganism testing apparatus using a fluorescence flow cytometry method, wherein
a microorganism detection unit is provided in a microorganism testing chip held by a movable stage,
the microorganism testing chip includes a mark disposed near the detection flow path, the mark being configured to increase an intensity of fluorescence by being irradiated with excitation light and having a predetermined positional relationship with the detection flow path in a direction of an optical axis of excitation light, and
processing for alignment of the detection flow path in the direction of the optical axis of the excitation light is performed based on fluorescence from the mark.

8. A method of aligning a microorganism detection unit with excitation light in a microorganism testing apparatus using a fluorescence flow cytometry method, wherein the microorganism detection unit is provided in a microorganism testing chip held by a movable stage,
the method comprising:
irradiating the microorganism detection unit with the excitation light; and
performing alignment of the microorganism detection unit in a direction of an optical axis of the excitation light by controlling and moving the movable stage in the direction of the optical axis of the excitation light such that the intensity of fluorescence detected from the microorganism detection unit reaches maximum.
